(19) United States
(12) Patent Application Publication
(10) Pub. No.: US 2023/0057383 A1
(43) Pub. Date: Feb. 23, 2023

(54) COMPOSITION AND METHODS FOR PREVENTION AND TREATMENT OF VASCULAR DISORDERS, NEURODEGENERATIVE DISEASE AND NEUROPATHIC DISORDERS

(71) Applicant: Mihan JAFARI JAVID, Tehran (IR)

(72) Inventors: Mihan Jafari Javid, Tehran (IR); Amir Dadgari, Tehran (IR)

(21) Appl. No.: 17/799,901

(22) PCT Filed: Mar. 21, 2020
(86) PCT No.: PCT/IB2020/052650

§ 371 Date: Aug. 15, 2022

(51) Int. Cl. A61K 33/22; C07K 16/22; A61K 36/28; A61K 33/14; A61K 33/06

(52) U.S. Cl. CPC A61K 33/22; C07K 16/22; A61K 36/28; A61K 33/14; A61K 33/06

(57) ABSTRACT

The present invention is directed to compositions and methods for the prevention and treatment of pathological vascular occlusive disorders and related medical conditions. The composition comprising boric acid, sodium chloride, potassium chloride, aluminum sulfate and chamomile.

### CROSS-REFERENCE TO RELATED APPLICATIONS

This application claims priority to the patent application serial number PCT/IB2020/052650 filed Mar. 21, 2020, which is incorporated herein by reference in its entirety.

### FIELD OF INVENTION

The present invention is directed to a composition and method for prevention and treatment of diseases related to pathological vascular disorders including pathological neovascularization of retina.

### BACKGROUND

Neovascularization is a process of formation of new blood vessels, usually in the form of functional microvascular networks. Under normal adult physiological conditions, microvascular networks are relatively quiescent, with neovascularization primarily limited to wound healing and steps in the female reproductive processes. So, neovascularization is a normal physiological process, however, pathological neovascularization is a critical component of diseases such as proliferative retinopathies, cancer, and rheumatoid arthritis. Particularly, pathological neovascularization is a critical component in proliferative retinopathies including retinopathy of prematurity, diabetic retinopathy, and the wet form of macular degeneration. Neovascularization is the hallmark of proliferative diabetic retinopathy. In proliferative retinopathies, cornea, retina, and choroid are the most common ocular tissues affected.

The normal vascular pattern of the ocular tissue is firmly controlled by the delicate balance between the pro-angiogenic and angiostatic molecules. For example, contributing factors to pathological neovascularization such as hypoxia, ischemia, and inflammation have been shown to disrupt the normal balance in the retinal expression of vascular endothelial growth factor (VEGF) and pigment epithelium-derived factor (PEDF). VEGF and PEDF are known to be the key angiogenic and angiostatic factors in retina respectively. Because of the absence of pericytes and basement membrane, the new vessels become more fragile and leakier leading to hemorrhage and edema, which interrupt light transmission ending by vision loss. Although the pathogenesis of proliferative retinopathies had been heavily studied, the exact underlying mechanism is not yet well understood.

Thus, compounds with roles in pathological neovascularization are considered potential targets for the treatment of the above conditions. There is a critical need for alternative and more effective treatments for prevention and treatment of pathological neovascularization.

### SUMMARY OF THE INVENTION

The following details present a simplified summary of the embodiments herein to provide a basic understanding of the several aspects of the embodiments herein. This summary is not an extensive overview of the embodiments herein. It is not intended to identify key/critical elements of the embodiments herein or to delineate the scope of the embodiments herein. Its sole purpose is to present the concepts of the embodiments herein in a simplified form as a prelude to the more detailed description that is presented later.

The principal objective of the present invention is therefore directed to a composition and method for the prevention and treatment of medical conditions having pathological vascular proliferations as an underlying cause.

It is another objective of the present invention to treat certain ophthalmic conditions, such as retinopathy of prematurity, diabetic retinopathy, age-related macular degeneration, and central retinal vein occlusion and any other kind of occlusive and/or inflammatory vasculopathy.

In one aspect, the present invention is directed to a composition for the prevention and treatment of pathological vascular disorders. The composition comprising boric acid, sodium chloride, potassium chloride, aluminum sulfate and chamomile extract.

In one aspect, the present invention is directed to a method for the prevention and treatment of pathological vascular proliferation. The method for the prevention and treatment of pathological vascular proliferation is particularly advantageous in the treatment of certain medical conditions including retinopathy of prematurity, diabetic retinopathy, age-related macular degeneration, central vein occlusion, atherosclerotic vascular disorders, tumors, and cancers. The method comprising a step of administering a composition comprising boric acid, sodium chloride, potassium chloride, aluminum sulfate and chamomile extract.

In one aspect, the present invention provides one or more formulations for the prevention and treatment of a medical condition having pathological vascular proliferation as an underlying cause. The formulations selected from a group consisting of topical eye drop, intra-vitreous injection, oral suspensions, tablets, capsules, topical gels, and intravenous injection.

These and other objects and advantages of the embodiments herein will become readily apparent from the following detailed description taken in conjunction with the accompanying drawings.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a table showing a composition, in accordance with an embodiment of the present invention.

FIG. 2 is a flow chart showing a method of preparation of the composition of FIG. 1, in accordance with an embodiment of the present invention.

### DETAILED DESCRIPTION

These embodiments are described in enough detail to enable those skilled in the art to practice the embodiments and it is to be understood that the logical, mechanical, and other changes may be made without departing from the scope of the embodiments. The following detailed description is therefore not to be taken in a limiting sense.

It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The compositions of the present invention can be formulated according to known methods for preparing pharmaceutically useful compositions. In general, the compositions of the present invention will be formulated such that an effective amount of the bioactive agent is combined with a suitable carrier in order to facilitate effective administration of the composition.

In accordance with the invention, pharmaceutical compositions comprise active agents, and one or more non-toxic, pharmaceutically acceptable carriers or diluents. Pharmaceutical carriers or excipients may contain inert ingredients that do not interact with the active ingredients, or ingredients that do interact with the active ingredients. In general, the formulations are prepared by uniformly and intimately bringing into association the active ingredient with a liquid or solid carrier.

The present invention is directed to a medical composition and a method for the prevention and treatment of one or more medical conditions having pathological vascular disorder as an underlying cause. The composition according to the present invention comprises a boric acid in a concentration for prevention and treatment of pathological vascular proliferation. Preferably, the boric acid can be present in the range of 0.1%-6% w/v. More, preferably, the boric acid can be present in the range of 0.1%-4% w/v. More, preferably, the boric acid can be present in the range of 0.1%-2% w/v. More, preferably, the boric acid can be present in the range of 1%-4% w/v. Boric acid (H3BO4), also known as hydrogen borate, Boracic acid, orthoboric acid, and acidum boricum, is a weak acid. It will be appreciated that boric acid is the active ingredient of the composition, other ingredients may also be present in the composition which facilitates the administration of the active ingredient. Such ingredients are generally inert and commonly known as excipients or carriers.

Sodium chloride is one of the most abundant minerals on earth and an essential nutrient for many of the biological processes to absorb and transport nutrients. Sodium chloride representing 1:1 ratio of sodium and chloride ions helps in maintaining the balance of the ionic channels. Potassium chloride has an essential role in the biological processes. Potassium chloride representing 1:1 ratio of Potassium and chloride ions helps in maintaining the balance of the ionic channels. Potassium aluminum sulfate, also known as, potash alum is the double sulfate of potassium and aluminum. The potassium aluminum sulfate can be used in the formulation in a concentration ranging from 0.01% to 0.1%. Chamomile is a well-known medicinal plant. Besides its anti-inflammatory effect, chamomile extract increases cell membrane permeability and enhances the efficacy of boric acid in the formulation by two or three-fold.

In one exemplary embodiment, the carrier can be selected from a group consisting of distilled water, normal saline, half saline, distilled water, boiled water, medical alcohols including ethyl alcohol, and hydrocortisone solution.

In one exemplary embodiment, the composition according to the present invention can be formulated as a topical composition, oral composition, parenteral composition, or an ocular composition. The topical compositions including topical gels and solution and topical patches. The oral compositions including tablets, capsules, suspensions, and syrups. The parenteral composition including intravenous injection, subcutaneous injection, sub-dermal, and intramuscular injection. The ocular composition including eye drops, eye gels, ocular implants, and intra-vitreous injection.

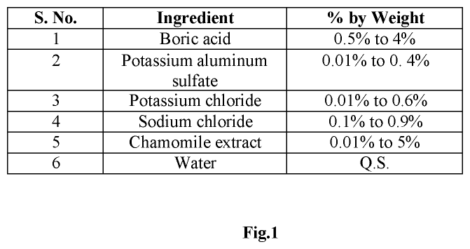

FIG. 1 shows an exemplary embodiment of the composition comprising boric acid in a range of 0.5-4% w/v, Potassium aluminum sulfate in a range of 0.01-0.4% w/v, Potassium chloride in a range of 0.01-0.6% w/v, Sodium chloride in a range of 0.1-0.9% w/v, Chamomile extract in a range of 0.01-5% w/v, and water Q. S.

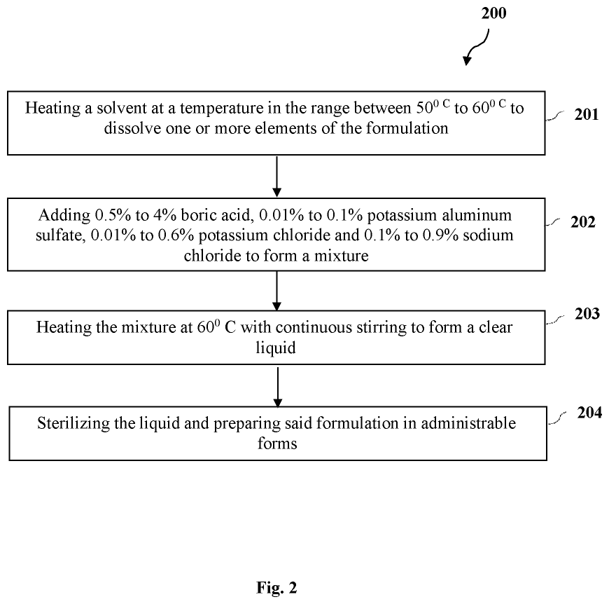

FIG. 2 shows an exemplary embodiment of a method 200 for preparing the composition shown in FIG. 1. At step 201, a predetermined quantity of Chamomile extract and water can be taken in a container. The solution can be heated to a temperature of 50-60 degrees Celsius. To the hot solution, at step 202, can be added the predetermined quantities of 0.5% to 4% boric acid, 0.01% to 0.1% potassium aluminum sulfate, 0.01% to 0.6% potassium chloride and 0.1% to 0.9% sodium chloride to form a mixture. The mixture, at step 203, can be continuously stirred while heating to dissolve the ingredients. Once a clear solution is obtained, the heating can be stopped, and the solution can be gradually cooled to room temperature. The solution, at step 204, can then be sterilized and formulated into the desired formulation.

In one exemplary embodiment, the active ingredient can be formulated into a composition in a neutral or salt form. Pharmaceutically acceptable salts include the salts derived from inorganic bases such as, for example, sodium, potassium, ammonium, calcium, or ferric hydroxides, and such organic bases as isopropyl amine, trimethylamine, histidine, procaine and the like.

In one exemplary embodiment, the composition can be formulated in a form suitable for intraocular injections. According to a first embodiment, the composition is an aqueous solution. Examples of aqueous solutions include, but are not limited to, a solution of boric acid in NaCl, preferably in 0.9% NaCl.

In one embodiment, sterile injectable solutions can be prepared by incorporating the active compound in the required amount in the appropriate solvent followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and freeze-drying techniques, which yield a powder of the active ingredient, plus any additional desired ingredient from a previously sterile-filtered solution thereof.

In one exemplary embodiment, the conditions related to pathological vascular proliferation include uveitis, choroiditis, retinochoroiditis, chorioretinitis, retinal degeneration, age-related macular degeneration, retinal detachment, retinal neovascularization, proliferative vitreoretinopathy, retinopathy of prematurity (ROP), diabetic retinopathy, posterior segment trauma, retinal vascular pathologies, endophthalmitis, macular edema, inflammatory pathologies of the retina, systemic pathologies with implications for the retina.

In a preferred embodiment, the conditions related to pathological vascular proliferation include retinopathy of prematurity, diabetic retinopathy, age-related macular degeneration, central vein occlusion, tumors, and cancers.

In one embodiment, the method according to the present invention comprises administration of the composition of the present invention in combination with other pharmacological therapies. Combination therapies with other medicaments targeting similar or distinct disease mechanisms have advantages of greater efficacy and safety relative to respective monotherapies with either specific medicament. In one case a VEGF inhibitor can be administered with the composition of the present invention. In another case, Bevacizumab or Aflibercept, or a combination thereof can be administered with the composition of the present invention.

What is claimed is:

1. A method of prevention and treatment of a medical condition having pathological vascular proliferation as an underlying cause, the method comprising administering to a patient, a composition, the composition comprising boric acid, sodium chloride, potassium chloride, aluminum sulfate and chamomile extract.

2. The method of claim 1, wherein the medical condition is retinopathy of prematurity.

3. The method of claim 1, wherein the medical condition is diabetic retinopathy

4. The method of claim 1, wherein the medical condition is age-related macular degeneration

5. The method of claim 1, wherein the medical condition is central retinal vein occlusion

6. The method of claim 1, wherein the medical condition is tumors or cancers.

7. The method of claim 1, wherein the medical condition is atherosclerotic vascular disorders in whole body vasculature.

8. The method of claim 1, wherein the boric acid is present in a concentration of 0.1%-6%.

9. The method of claim 1, wherein the composition is administered as an ocular formulation to an eye, the ocular formulation selected from a group consisting of eye drops, eye gels, ocular implants, and intravitreal injection.

10. The method of claim 1, wherein the composition is administered as an oral formulation, the oral formulation selected from a group consisting of tablet, capsule, suspension, and syrup.

11. The method of claim 1, wherein the composition is administered as a parenteral formulation, the parenteral formulation selected from a group consisting of intravenous injection, intramuscular injection, and subcutaneous injection.

12. The method of claim 1, wherein the composition is administered as a topical spray or a topical patch for the medical conditions of atherosclerotic disorders and vasculitis.

13. The method of claim 1, wherein the method further comprises a step of administering a VEGF inhibitor.

14. The method of claim 1, wherein the method further comprises a step of administering Bevacizumab or Aflibercept, or a combination thereof.

15. A method of prevention and treatment of an ischemia related vascular disorders, the method comprising administering to a patient, a composition, the composition comprises a boric acid, wherein the composition prevents and treats the ischemia related vascular disorders.

16. The method of claim 15, wherein the method further comprises a step of administering a VEGF inhibitor.

17. The method of claim 15, wherein the boric acid is present in a concentration of 0.1%-6%.

18. A composition comprising:
boric acid in a range of 0.5-4% w/v;
potassium aluminum sulfate in a range of 0.01-0.4% w/v;
potassium chloride in a range of 0.01-0.6% w/v;
sodium chloride in a range of 0.1-0.9% w/v;
chamomile extract in a range of 0.01-5% w/v; and
water Q.S.
